(19) United States
(12) Patent Application Publication
(10) Pub. No.: US 2026/0027246 A1
(43) Pub. Date: Jan. 29, 2026

(54) INACTIVATION APPARATUS

(71) Applicant: Ushio Denki Kabushiki Kaisha, Tokyo (JP)

(72) Inventor: Hideaki YAGYU, Tokyo (JP)

(73) Assignee: Ushio Denki Kabushiki Kaisha, Tokyo (JP)

(21) Appl. No.: 18/998,442

(22) PCT Filed: Jun. 28, 2023
(86) PCT No.: PCT/JP2023/023888

§ 371 Date: Jan. 24, 2025

(30) Foreign Application Priority Data
Jul. 26, 2022 (JP) 2022-118433

(51) Int. Cl. A61L 2/10; A61L 2/00; A61L 2/26; H01J 61/12; H01J 61/16; H01J 61/40; H01J 65/00

(52) U.S. Cl. CPC A61L 2/10; A61L 2/0094; A61L 2/26; H01J 61/125; H01J 61/16; H01J 61/40; H01J 65/00; A61L 2209/12

(57) ABSTRACT

The inactivation apparatus includes: an excimer lamp that includes a luminous tube, and a pair of electrodes, and generates ultraviolet light having a main emission wavelength band within a range of 190 nm or more and less than 240 nm in the luminous tube when a voltage is applied between the pair of electrodes; and an optical filter into which ultraviolet light generated by the excimer lamp is incident, the optical filter transmitting ultraviolet light having a wavelength within a range of 190 nm or more and less than 240 nm and substantially not transmitting ultraviolet light having a wavelength within a range of 240 nm or more and less than 280 nm, in which a sealing pressure ratio of the halogen gas to the noble gas in the luminous gas sealed in the luminous tube is 2% or more and less than 5%.

### TECHNICAL FIELD

The present invention relates to an inactivation apparatus for bacteria or viruses and particularly to an inactivation apparatus for bacteria or viruses using ultraviolet light.

### BACKGROUND ART

Conventionally, technologies for inactivating bacteria or viruses by irradiating bacteria or viruses with ultraviolet light have been known. Deoxyribonucleic acid (DNA) exhibits the highest absorption characteristics around a wavelength of 260 nm, and therefore in many cases, ultraviolet light that is emitted from a light source such as a low-pressure mercury lamp and has a wavelength of around 254 nm is used. The method of inactivating bacteria and viruses by the ultraviolet light has an advantage that sterilization treatment can be performed only by irradiating a target space or an object with ultraviolet light without spraying a chemical agent or the like.

The ultraviolet light has a wavelength band having a high risk of affecting the human body and a wavelength band having a low risk of affecting the human body. Therefore, methods and apparatuses for inactivating bacteria and viruses that are present in the space by ultraviolet light in a wavelength band having a low risk of affecting the human body have been studied. For example, Patent Document 1 indicated below gives a description of a sterilization device (inactivation apparatus) that uses ultraviolet light in a wavelength range of 190 nm to 230 nm, which has an extremely small influence on the human body.

### PRIOR ART DOCUMENT

### Patent Document

Patent Document 1: JP-B2-6025756

### SUMMARY OF THE INVENTION

### Problems to be Solved by the Invention

In recent years, the influence of ultraviolet light on the human body has been researched and verified in a progressive manner, and it has been confirmed that the ultraviolet light is easily absorbed in a skin surface layer and corneal epithelium and is improved in safety with a decrease in wavelength. In particular, it has been confirmed that the ultraviolet light with a wavelength of shorter than 240 nm has a low risk of affecting the human body.

In addition, an inactivation apparatus using the ultraviolet light in a wavelength band having a small influence on the human body has attracted particular attention due to the influence of the recent spread of coronavirus infection, and for introduction to a space or the like where people frequently come and go, an apparatus that can perform inactivation treatment more safely and more efficiently is expected.

In view of the above problems, an object of the present invention is to provide an inactivation apparatus that maintains or suppresses the intensity of ultraviolet light in a wavelength band that possibly affect the human body while improving the intensity of ultraviolet light in a wavelength band that has an extremely small influence on the human body.

### Means for Solving the Problems

The inactivation apparatus according to the present invention includes:
an excimer lamp that includes a luminous tube in which a luminous gas containing a noble gas and a halogen gas is sealed, and a pair of electrodes, and generates ultraviolet light having a main emission wavelength band within a range of 190 nm or more and less than 240 nm in the luminous tube when a voltage is applied between the pair of electrodes; and
an optical filter into which ultraviolet light generated by the excimer lamp is incident, the optical filter transmitting ultraviolet light having a wavelength within a range of 190 nm or more and less than 240 nm and substantially not transmitting ultraviolet light having a wavelength within a range of 240 nm or more and less than 280 nm, in which
the luminous gas sealed in the luminous tube has a ratio of a sealing pressure of the halogen gas to a sealing pressure of the noble gas of 2% or more and less than 5%.

“Inactivation” herein refers to a concept that includes killing bacteria or viruses or making infectivity or toxicity thereof lost, and “bacteria” refer to microorganisms such as germs or fungi (mold). Hereinafter, in some cases, the “bacteria or viruses” are collectively referred to as “pathogens”.

The “main emission wavelength band” herein refers to a wavelength band across which an intensity spectrum of light generated in the luminous tube of the excimer lamp indicates a light intensity of 10% or more of a peak intensity of the intensity spectrum.

In addition, “transmitting ultraviolet light” herein means that regarding ultraviolet light incident at an incident angle of 0° and emitted at an emission angle of 0°, an intensity of 10% or more is maintained with respect to the intensity of a peak wavelength. Note that the intensity of ultraviolet light in the wavelength band to be transmitted through the optical filter is preferably maintained at 10% or more, more preferably 20% or more with respect to the intensity of the peak wavelength. Further, “substantially not transmitting ultraviolet light” means that the intensity is suppressed down to 5% or less of the intensity at the peak wavelength, regarding the ultraviolet light that is incident at an incident angle of 0° and emitted at an emission angle of 0°. Note that the intensity of ultraviolet light in the wavelength band to be suppressed by the optical filter is preferably suppressed to 2% or less and more preferably suppressed to 1% or less of the intensity at the peak wavelength.

First, characteristics of an excimer lamp including a luminous tube in which a luminous gas containing noble gas atoms (Ng) and halogen atoms (X) is sealed will be described.

In the excimer lamp, when a voltage of a predetermined threshold value or higher is applied between a pair of electrodes, discharge occurs in the luminous tube. Then, the noble gas atoms and the halogen atoms contained in the luminous gas are ionized or excited by this discharge, and excited complexes are formed in the luminous tube as shown in the following formula (1). The excited complex is an extremely unstable molecule, and immediately after being formed, dissociates into a noble gas atom and a halogen atom as shown in the following formula (2). Then, when this dissociation occurs, light (also referred to as “excimer light”) corresponding to the magnitude of energy to be released is generated. (*) in the following formulas indicates an excited state. Note that each reaction formula shown below is a formula showing a representative part of reactions involved in the generation of excimer light among various reactions generated in the luminous tube.

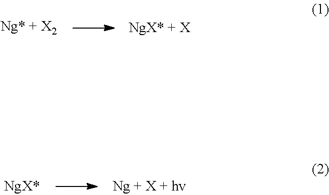

The light generated by the reactions represented by the above formulas (1) and (2) is generally light belonging to a main emission wavelength band among the light generated in the luminous tube.

In the luminous tube, in some cases, the reactions represented by the following formulas (3) and (4) occur together with the reactions represented by the above formulas (1) and (2) depending on the combination of atoms contained in the luminous gas and the sealing pressure.

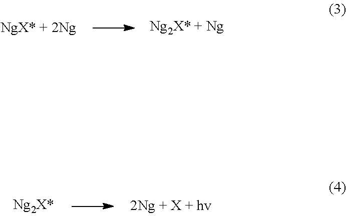

The light generated by the reactions represented by the formulas (3) and (4) generally has energy smaller than that of the light in the main emission wavelength band among the light generated in the luminous tube, and is light on the longer wavelength side than the main emission wavelength band. For example, in the case of an excimer lamp in which the noble gas is krypton (Kr) and the halogen gas is chlorine (Cl), light in the vicinity of 222 nm is generated by the reactions represented by the formulas (3) and (4) above, and light in the vicinity of 315 nm is generated by the reactions represented by the formulas (3) and (4) above.

The ultraviolet light having a wavelength in the vicinity of 315 nm is light in a wavelength band that causes sunburn or the like when the human body is irradiated with the ultraviolet light. However, regarding the ultraviolet light emitted from the excimer lamp having the above configuration, the intensity of ultraviolet light having a wavelength in the vicinity of 315 nm is very weak with respect to the intensity of light belonging to the main emission wavelength band.

In addition, even if the excimer lamp is configured to emit light whose main emission wavelength band is within a range of 190 nm or more and less than 240 nm, in many cases, ultraviolet light within a range of 240 nm or more and 280 nm, which is a wavelength band particularly harmful to the human body, is inevitably generated. Such ultraviolet light having a wavelength in the range of 240 nm or more and 280 nm is often prevented by providing an optical filter that does not substantially transmit ultraviolet light in the concerned wavelength band.

Here, the actual situation and trend of the inactivation apparatus using the ultraviolet light will be described. As described above, the inactivation apparatus using the ultraviolet light in a wavelength band having an extremely small influence on the human body can be expected to have an effect of suppressing contact infection via an object surface and infection via aerosol present in the space. Therefore, introduction of the inactivation apparatus into a space where people frequently come and go or a space where people work for a long time has been studied.

However, although the ultraviolet light with wavelengths of 190 nm or more and less than 240 nm has an extremely small influence on the human body compared to the ultraviolet light emitted from low-pressure mercury lamps, a regulation value concerning an integrated irradiation dose to the human body is prescribed in consideration of safety. At the time of filing of the present application, it is recommended that the integrated irradiation dose of the ultraviolet light irradiated on the human body is set within the regulation value (threshold limit value) specified by American Conference of Governmental Industrial Hygienists (ACGIH). For example, the threshold limit value for the integrated irradiation dose of the ultraviolet light with a wavelength of 222 nm a day (eight hours) is specified to be 22 mJ/cm². Note that, the threshold limit value herein is the current numerical value and is a numerical value is possibly altered in the future. In addition to the above case, it is desirable to set a predetermined upper limit value for the integrated irradiation dose of ultraviolet light irradiated to the human body from the perspective of safe operation.

Therefore, the inactivation apparatus that is assumed to irradiate a space or the like where people come and go with ultraviolet light is required to be able to efficiently perform the inactivation treatment on a space to be treated or an object to be treated while complying with the regulation value of the integrated irradiation dose of ultraviolet light described above.

As described above, in the excimer lamp that emits the ultraviolet light having a main emission wavelength band within a range of 190 nm or more and less than 240 nm, light having a wavelength in the vicinity of 315 nm is much weaker than the intensity of ultraviolet light belonging to the main emission wavelength band. In addition, the intensity of ultraviolet light having a wavelength in the range of 240 nm or more and 280 nm is sufficiently reduced by the optical filter.

Therefore, when the excimer lamp that emits the ultraviolet light having a main emission wavelength band within a range of 190 nm or more and less than 240 nm is used and the inactivation treatment is performed in compliance with the threshold limit value at the current time point, the influence on the human body hardly becomes a problem.

However, because the recent spread of coronavirus infection has attracted attention to the inactivation treatment of bacteria and the like by ultraviolet light and the safety of the ultraviolet light in a specific wavelength band has been confirmed by various verification experiments, currently, relaxation of the threshold limit value of the integrated irradiation dose on humans has been studied for the ultraviolet light in some wavelength bands.

In view of the above circumstances, in order to more efficiently inactivate a target space and a target object, the inactivation apparatus using the ultraviolet light is expected to be used in the future in a usage mode such as irradiating the target space and the target object with the ultraviolet light having a higher intensity or constantly irradiating the target space in a space where people come and go.

Therefore, in the case where the inactivation apparatus is introduced into a space where people frequently come and go, it is expected that the risk of human health damage will become a problem in the future by reviewing the threshold limit value even if the risk is not a problem at the current time point. That is, in view of the possibility that the regulation value of the integrated irradiation dose is relaxed in the future, it is expected that, for the inactivation apparatus using the ultraviolet light, a technique is required to increase the intensity of ultraviolet light in the wavelength band that has a small influence on the human body, and meanwhile, maintain or reduce the intensity of ultraviolet light in the wavelength band that affects the human body.

Regarding the ultraviolet light having a wavelength in the range of 240 nm or more and less than 280 nm, because the influence on the human body is particularly large as compared with the ultraviolet light belonging to other wavelength ranges, it is conceivable that measures are taken intensively by devising the structure, material, and the like of the optical filter.

However, it is very difficult to configure the optical filter so as to satisfy desired transmittance characteristics over the entire wavelength band of ultraviolet light. In the optical filter, when the transmittance of a specific wavelength band is made to decrease, the transmittance of light in other wavelength bands inevitably increase in some cases.

Therefore, in the conventional inactivation apparatus, when the intensity of light emitted from the excimer lamp is increased and the transmittance of the ultraviolet light having a wavelength of 240 nm or more and less than 280 nm is reduced as much as possible, there is a risk that the ultraviolet light having a wavelength of 280 nm or more, particularly the ultraviolet light having a wavelength in the vicinity of 300 nm as described above is emitted with high intensity.

Therefore, the present inventor has focused on the fact that light generated in the luminous tube of the excimer lamp is generated on the basis of the reactions represented by the above formulas (1) to (4), and studied a method of controlling the spectrum of ultraviolet light by adjusting the sealing pressure of the noble gas and the halogen gas of the luminous gas sealed in the luminous tube.

The present inventor conducted a verification experiment to confirm how the spectrum of light generated in the luminous tube of the excimer lamp changes with respect to a change in the ratio of the sealing pressure of the noble gas and the halogen gas in the luminous gas sealed in the luminous tube. Details of the verification will be described later in the section of “MODE FOR CARRYING OUT THE INVENTION”.

According to the results of the verification experiment, it is confirmed that the ratio of the sealing pressure of the halogen gas to the sealing pressure of the noble gas in the luminous gas (hereinafter, also referred to as “sealing pressure ratio”) is preferably 2% or more and less than 5%.

As described above, by having the above configuration, regarding the relative intensity with respect to the peak intensity of ultraviolet light emitted through the optical filter, the intensity of ultraviolet light on the longer wavelength side than the main emission wavelength band can be reduced without greatly increasing the intensity of ultraviolet light in the wavelength range including the wavelength of 240 nm or more and less than 280 nm, which a wavelength band harmful to the human body. That is, the intensity of ultraviolet light in the wavelength band harmful to the human body can be maintained or reduced while the light intensity of ultraviolet light in the main emission wavelength band belonging to the wavelength band of 190 nm or more and less than 240 nm is increased.

In the inactivation apparatus described above,

the optical filter may have a band that transmits ultraviolet light in at least a part of a range of a wavelength of 280 nm or more and less than 320 nm.

A dielectric multilayer film filter can adjust the wavelength band of the ultraviolet light to be transmitted by adjusting the film thickness and the number of layers. For this reason, the dielectric multilayer film filter is often used as a band pass filter for ultraviolet light. However, in the dielectric multilayer film filter, there is a case where both a wavelength band to be transmitted and a wavelength band not to be substantially transmitted cannot be designed according to a desired specification. For example, when the filter is configured to allow the ultraviolet light belonging to a wavelength band of 190 nm or more and less than 240 nm to be transmitted and the ultraviolet light in a wavelength band of 240 nm or more and less than 280 nm not to be substantially transmitted, a band for transmitting light appears in a band of a wavelength of 280 nm or more in many cases (see FIG. 6A).

The dielectric multilayer film filter is characterized in that, when a bandwidth of a wavelength band that is not to be substantially transmitted is made to increase, the transmittance for ultraviolet light that needs to be transmitted and having a wavelength of 190 nm or more and less than 240 nm decreases. In particular, it is not realistic to configure the filter so as not to substantially transmit the light of the entire band belonging to the ultraviolet light region having a wavelength of 280 nm or more, because the transmittance with respect to the ultraviolet light having a wavelength of 190 nm or more and less than 240 nm is greatly reduced.

Therefore, by adopting the inactivation apparatus having the above configuration, the irradiation of ultraviolet light in a wavelength band of 240 nm or more and less than 280 nm with respect to a person can be suppressed, and meanwhile, the irradiation of the ultraviolet light that is generated by the reactions of the formulas (3) and (4) and that possibly affect the human body can also be suppressed. Note that, from the viewpoint of further improving safety, the optical filter is preferably a dielectric multilayer film filter that does not substantially transmit the ultraviolet light having a wavelength in a range of 280 nm or more and less than 350 nm.

In the inactivation apparatus described above, the luminous gas may be a mixed gas containing krypton (Kr) and chlorine (Cl).

Furthermore, in the inactivation apparatus described above, the luminous gas may be a mixed gas containing krypton (Kr) and bromine (Br).

Note that a product covered by the present invention can provide sterilization and virus inactivation performance intrinsic to ultraviolet light without causing erythema or keratitis on the skin or eyes of a human or an animal. In particular, taking advantage of characteristics of being able to be used in an environment where a human is present in contrast to conventional ultraviolet light sources, the product can be installed in an indoor or outdoor environment where a human is present to allow the entire environment to be irradiated and provide virus inhibition and bacteria elimination in the air and on surfaces of members installed in the environment.

This accords with Goal 3 “Ensure healthy lives and promote well-being for all at all ages” included in the Sustainable Development Goals (SDGs) led by the United Nations, and will greatly contribute to Target 3.3 “By 2030, end the epidemics of AIDS, tuberculosis, malaria and neglected tropical diseases and combat hepatitis, water-borne diseases and other communicable diseases”.

### Effect of the Invention

According to the present invention, it is possible to achieve an inactivation apparatus that maintains or suppresses the intensity of ultraviolet light in a wavelength band that possibly affect the human body while improving the intensity of ultraviolet light in a wavelength band that has an extremely small influence on the human body.

### BRIEF DESCRIPTION OF THE DRAWINGS

FIG. 1 is a view schematically illustrating an appearance of an embodiment of an inactivation apparatus.

FIG. 2 is a cross-sectional view of the inactivation apparatus in FIG. 1 as viewed in an X direction.

FIG. 3 is an enlarged view of the periphery of an excimer lamp in FIG. 2.

FIG. 4 is an enlarged view of the excimer lamp in FIG. 2.

FIG. 5 is a graph illustrating an example of a spectrum of ultraviolet light generated in a luminous tube of the excimer lamp.

FIG. 6A is a graph illustrating transmittance characteristics of an optical filter in the embodiment.

FIG. 6B is a graph illustrating an example of a spectrum of light emitted from the excimer lamp and passing through the optical filter.

FIG. 7 is a graph plotting relative intensity in a wavelength range of 280 nm or more and less than 320 nm for every excimer lamp of an experimental sample.

FIG. 8A is a relative intensity spectrum in a wavelength range of 230 nm to 280 nm of ultraviolet light emitted from the excimer lamp of the experimental sample.

FIG. 8B is a relative intensity spectrum in a wavelength range of 250 nm to 400 nm of ultraviolet light emitted from the excimer lamp of the experimental sample.

FIG. 9 is a view schematically illustrating an external appearance of an inactivation apparatus according to an embodiment.

FIG. 10 is a view of the inactivation apparatus in FIG. 9 as viewed from +Z side.

FIG. 11 is a cross-sectional view of the inactivation apparatus in FIG. 9 as viewed in an X direction.

FIG. 12 is an enlarged view of the periphery of an excimer lamp in FIG. 11.

### MODE FOR CARRYING OUT THE INVENTION

### First Embodiment

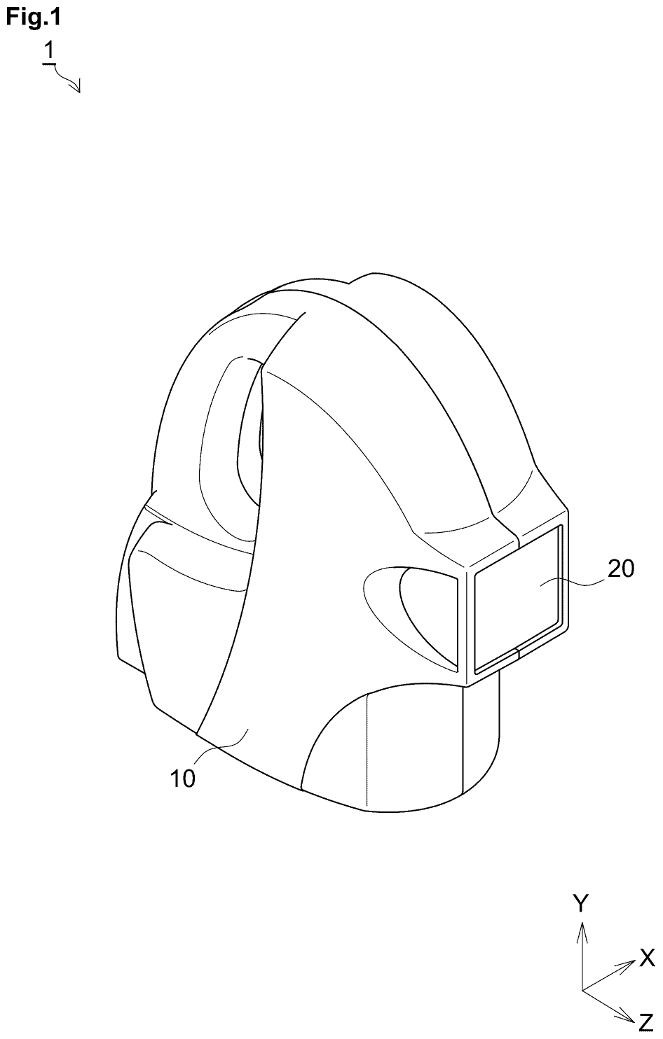

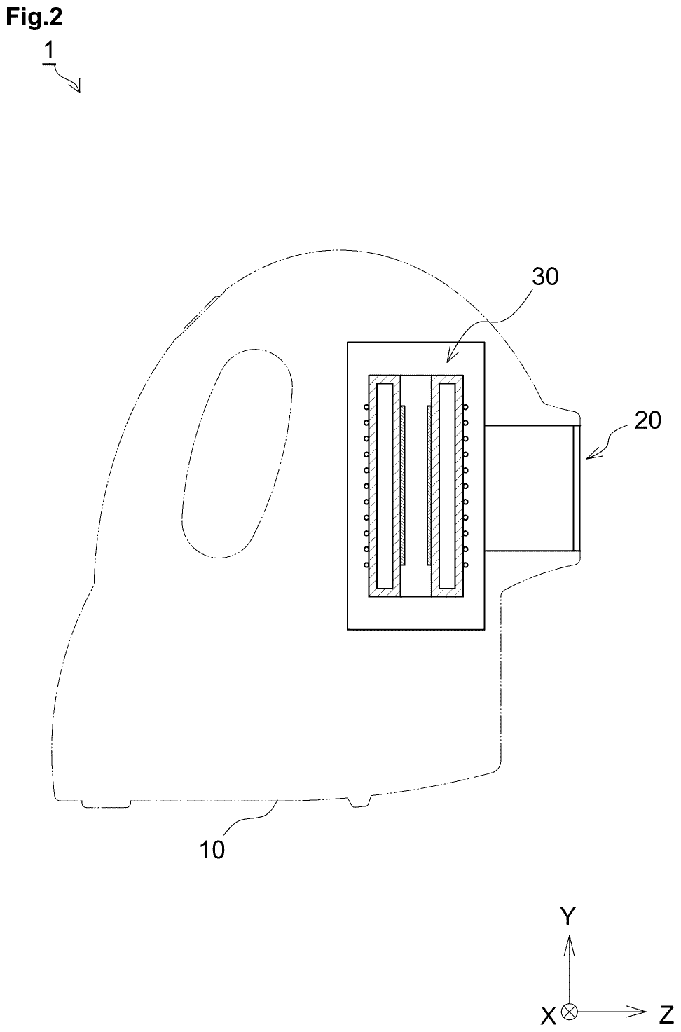

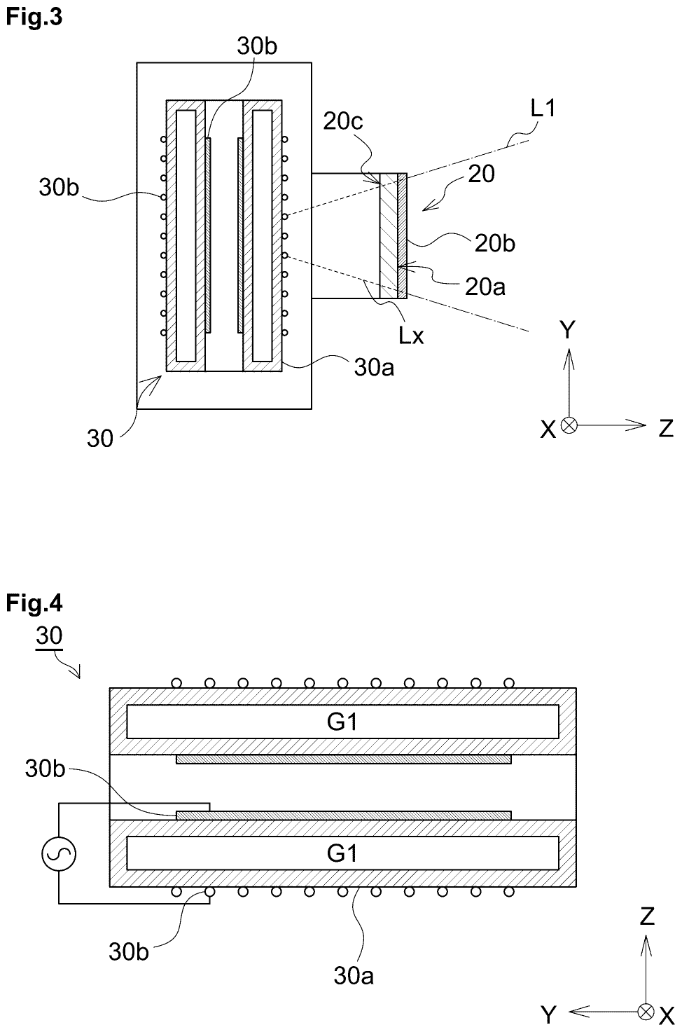

FIG. 1 is a schematic view illustrating an external appearance of an inactivation apparatus 1 according to an embodiment, and FIG. 2 is a cross-sectional view of the inactivation apparatus 1 in FIG. 1 as viewed in the X direction. FIG. 3 is an enlarged view of the periphery of an excimer lamp 30 in FIG. 2, and FIG. 4 is an enlarged view of the excimer lamp 30 in FIG. 2. The inactivation apparatus 1 of the first embodiment includes a housing 10 and a light transmission window 20 as illustrated in FIG. 1, and the excimer lamp 30 is accommodated in the housing 10 as illustrated in FIG. 2.

The first embodiment of the inactivation apparatus 1 has a shape in which a use mode in which the sterilization treatment is locally performed by irradiating the human skin with ultraviolet light is assumed. Note that the embodiment of the inactivation apparatus 1 is not limited to this embodiment, and it is naturally assumed that the inactivation apparatus 1 is fixed to a ceiling, a wall surface, a pole, or the like in a predetermined partitioned space to sterilize the inside of the space. In addition, the shape of the inactivation apparatus 1, the arrangement position of each member, and the like are designed in an optional shape according to the use mode.

In the following description, as illustrated in FIG. 2, a direction in which the excimer lamp 30 extends is referred to as a Y direction, a direction in which the light transmission window 20 and the excimer lamp 30 face each other is referred to as a Z direction, and a direction orthogonal to the Y direction and the Z direction is referred to as an X direction.

In addition, in a case where positive and negative orientations are distinguished from each other in expressing directions, positive and negative signs are added and the directions are expressed as the “+Z direction” and the “−Z direction”, and in a case where positive and negative orientations are not distinguished from each other in expressing directions, the direction is simply described as the “Z direction”. In the inactivation apparatus 1 in the first embodiment, as illustrated in FIG. 3, a direction in which ultraviolet light is extracted corresponds to “+Z direction”.

As illustrated in FIG. 3, the excimer lamp 30 of the first embodiment is an excimer lamp including a luminous tube 30a and a pair of electrodes 30b radially face each other with the luminous tube 30a interposed therebetween. Note that the excimer lamp 30 in the first embodiment is an excimer lamp in which the luminous tube 30a has a cylindrical shape and is also referred to as a double tube shape. In the excimer lamp 30 having such a shape, in order to extract ultraviolet light generated inside the luminous tube 30a to the outside, the outer electrode 30b is constituted of a metal wire processed into a mesh shape.

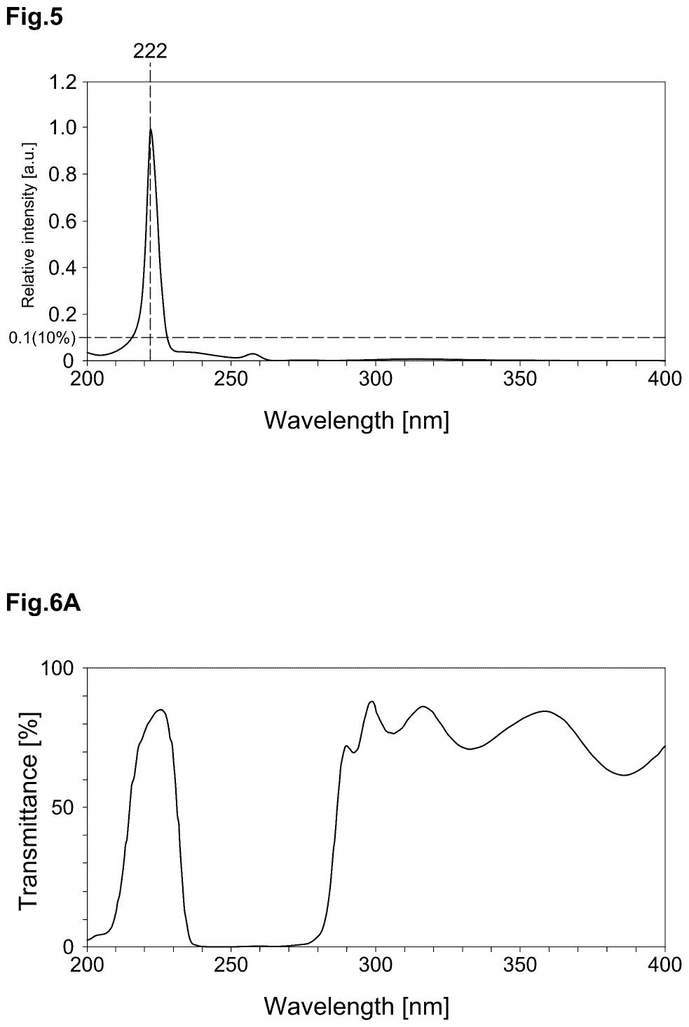

FIG. 5 is a graph illustrating an example of a spectrum of the ultraviolet light generated in the luminous tube 30a of the excimer lamp 30. In the graph illustrated in FIG. 5, the vertical axis represents the relative intensity of the time when the peak intensity (light intensity at a wavelength of 222 nm) is 100%, and the horizontal axis represents the wavelength. In the excimer lamp 30 of the first embodiment, a luminous gas G1 containing a mixed gas of krypton (Kr) gas and chlorine (Cl) gas is sealed in the luminous tube 30a, and when a voltage is applied between the electrodes (30b, 30b), the ultraviolet light having a spectrum as illustrated in FIG. 5 is generated in the luminous tube 30a. The graph illustrated in FIG. 5 is a graph measured by using a spectral radiometer (specifically, “USR-45DA” manufactured by Ushio Inc.).

The luminous gas G1 sealed in the luminous tube 30a contains argon (Ar) gas as a buffer gas together with the krypton gas and the chlorine gas so that the entire sealing pressure becomes 200 torr.

The main emission wavelength band of the excimer lamp 30 in the first embodiment is 216 nm to 223 nm as illustrated in FIG. 5. The main emission wavelength band of the excimer lamp 30 is preferably within a wavelength range of 190 nm or more and 240 nm or less, and more preferably within a wavelength range of 200 nm or more and 230 nm or less, in which the influence on the human body is small and the effect of the inactivation treatment is recognized.

In the present embodiment, the luminous gas G1 sealed in the luminous tube 30a is adjusted to have a ratio of a sealing pressure (P_{Cl}) of chlorine gas to a sealing pressure (P_{Kr}) of krypton gas, which is a ratio (P_{Cl}/P_{Kr}), of 3.33%.

The sealing pressure (P_{Kr}, P_{Cl}) of each gas contained in the luminous gas G1 sealed in the luminous tube 30a is measured by breaking the luminous tube 30a in a state of being accommodated in a vacuum container and using gas chromatography.

The light transmission window 20 is a light emission window through which the ultraviolet light emitted from the excimer lamp 30 is extracted to the outside of the housing 10. In the light transmission window 20 of the first embodiment, an optical filter 20b including a multilayer dielectric film is formed on a principal surface 20a.

In the inactivation apparatus 1, the ultraviolet light having the spectrum as illustrated in FIG. 5 passes through the optical filter 20b, which is described later with reference to FIG. 3, and then is extracted to the outside of the housing 10 from the light transmission window 20. Note that, in FIG. 4, the ultraviolet light that has been generated by the excimer lamp 30 is denoted by “ultraviolet light Lx” and the ultraviolet light that has passed through the light transmission window 20 and that has been extracted out of the inactivation apparatus 1 is denoted by “ultraviolet light L1” to distinguish both states of the ultraviolet light from each other. Similar expressions are used hereinafter as appropriate.

The light transmission window 20 is constituted of material that allows transmission of the ultraviolet light in a wavelength band from 190 nm or more to less than 240 nm. The specific material that can be adopted for the light transmission window 20 includes, for example, a ceramic-based material such as silica glass, borosilicate glass, sapphire, magnesium fluoride material, calcium fluoride material, lithium fluoride material, and barium fluoride material, or a resin-based material such as a silicon resin and a fluororesin.

In addition, as illustrated in FIG. 3, the optical filter 20b of the first embodiment is formed on the principal surface 20a of the light transmission window 20, but may be formed on a principal surface 20c on the opposite side of the principal surface 20a of the light transmission window 20. Furthermore, in the case where the light transmission window 20 has a configuration in which the optical filter 20b can be mounted alone without requiring a glass plate or the like, the light transmission window 20 may be constituted only of the optical filter 20b.

In the first embodiment, a length in a tube axis direction (the Y direction) of the luminous tube 30a of the excimer lamp 30 is 120 mm, a distance between the excimer lamp 30 and the optical filter 20b is 40 mm, and a size of the optical filter 20b is (X, Y)=(50 mm, 50 mm). Note that each size configuration described herein is merely an example, and each size is optional.

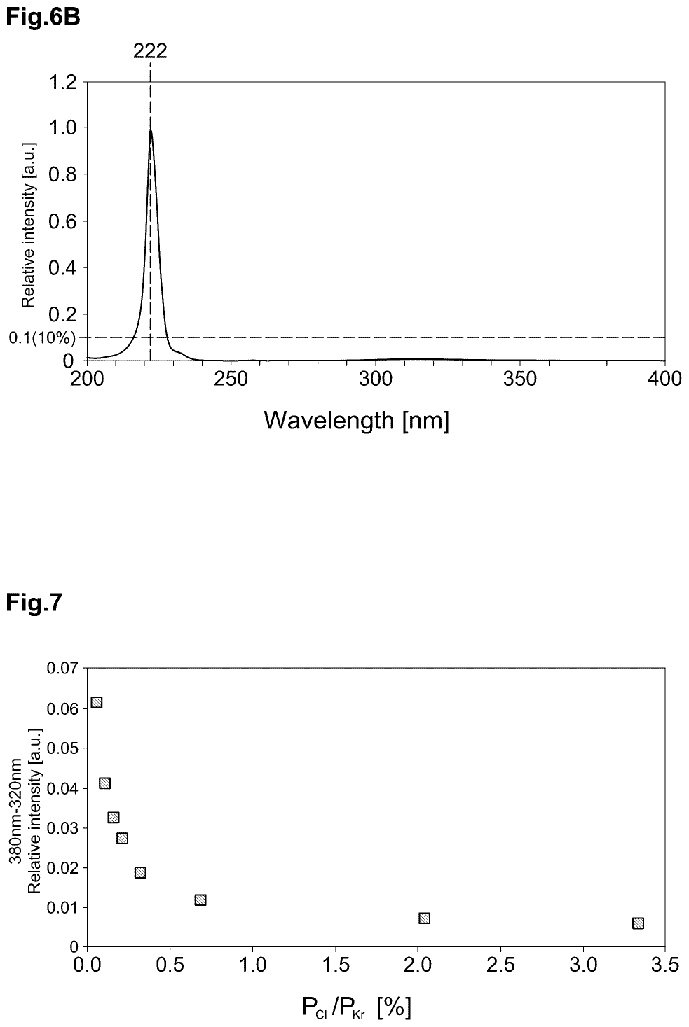

FIG. 6A is a graph illustrating transmittance characteristics of the optical filter 20b according to the first embodiment. In the graph illustrated in FIG. 6A, the vertical axis represents the transmittance of the optical filter 20b, and the horizontal axis represents the wavelength. Note that the graph in FIG. 6A is a graph obtained by measuring a spectral spectrum of a light beam incident on the optical filter 20b at an incident angle of 0° and a spectral spectrum of a light beam emitted from the optical filter 20b at an emission angle of 0° by using a spectrophotometer (specifically, “V-7200” manufactured by JASCO Corporation).

The optical filter 20b in the first embodiment is constituted of a dielectric multilayer film, and as illustrated in FIG. 6A, is configured to transmit the ultraviolet light having a wavelength of 210 nm or more and less than 240 nm and substantially not transmit the ultraviolet light having a wavelength of 240 nm or more and less than 280 nm. Furthermore, as illustrated in FIG. 6A, the optical filter 20b transmits the ultraviolet light having a wavelength of 280 nm or more and less than 400 nm.

FIG. 6B is a graph illustrating an example of a spectrum of light emitted from the excimer lamp 30 and passing through the optical filter 20b. In the spectrum illustrated in FIG. 6B, it is confirmed that the intensity in a wavelength range of 240 nm or more and less than 280 nm is reduced by the optical filter 20b as compared with the spectrum in the graph illustrated in FIG. 5. Note that, similarly to FIG. 5, the graph illustrated in FIG. 6B is a graph measured by using a spectral radiometer (specifically, “USR-45DA” manufactured by Ushio Inc.).

The optical filter 20b constituted of the dielectric multilayer film can adjust the wavelength band to be transmitted and the wavelength band not to be substantially transmitted by finely adjusting the film thickness of each film constituting the dielectric multilayer film. Examples of the material constituting each layer of the dielectric multilayer film include silica (SiO₂), hafnia (HfO₂), alumina (Al₂O₃), titania (TiO₂), and zirconia (ZrO₂).

### [Verification Experiment]

Here, a verification experiment for confirming the relationship between the intensity spectrum of the ultraviolet light Lx emitted from the excimer lamp 30 and the sealing pressure ratio (P_{Cl}/P_{Kr}) between the noble gas and the halogen gas contained in the luminous gas G1 sealed in the luminous tube 30a of the excimer lamp 30 has been performed, and thus, the experiment will be described.

### (Verification Method)

The sealing pressure (P_{Kr}) of krypton (Kr) gas, the sealing pressure (P_{Cl}) of chlorine (Cl) gas, and the sealing pressure ratio (P_{Cl}/P_{Kr}) included in the luminous gas G1 sealed in the luminous tube 30a of the excimer lamp 30 were set as shown in Table 1 below. As described above, the luminous gas G1 sealed in the luminous tube 30a contains argon (Ar) gas as a buffer gas, and the entire sealing pressure is adjusted to 200 torr in all the samples.

TABLE 1

| Samples | P_{Kr}(torr) | P_{Cl}(torr) | P_{Cl}/P_{Kr} |
|---|---|---|---|
| Example 1 | 120 | 4.0 | 3.33% |
| Example 2 | 142 | 2.9 | 2.04% |
| Comparative Example 1 | 176 | 1.2 | 0.68% |
| Comparative Example 2 | 188 | 0.6 | 0.32% |
| Comparative Example 3 | 196 | 0.2 | 0.10% |

The light intensity was measured at a position 50 mm away from the luminous tube 30a of the excimer lamp 30. Note that, because the purpose of the present verification is to confirm the correlation characteristics between the sealing pressure ratio (P_{Cl}/P_{Kr}) and the spectrum of the ultraviolet light emitted from the excimer lamp 30, the present verification was performed without the optical filter 20b.

### (Verification Result)

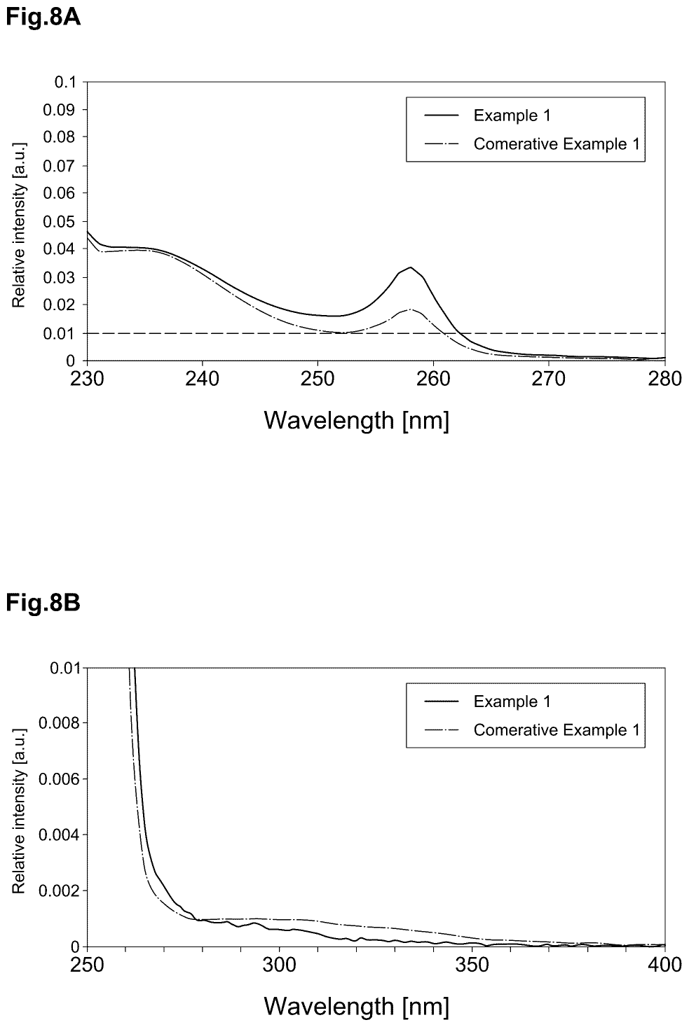

FIG. 7 is a graph plotting the relative intensity in a wavelength range of 280 nm or more and less than 320 nm for every excimer lamp of the sample shown in the above Table 1. Note that the relative intensity illustrated in FIG. 7 is a light intensity integrated value in a wavelength range of 280 nm or more and less than 320 nm at the time when the light intensity integrated value in a wavelength range of 222 nm±5 nm is normalized as 1. FIG. 8A is a relative intensity spectrum in a wavelength range of 230 nm to 280 nm of the ultraviolet light Lx emitted from the samples of Example 1 and Comparative Example 1 shown in Table 1, and FIG. 8B is a relative intensity spectrum in a wavelength range of 250 nm to 400 nm of the ultraviolet light Lx emitted from the samples of Example 1 and Comparative Example 1 shown in Table 1. Note that the relative intensity spectrum of the ultraviolet light emitted from the excimer lamp 30 of Example 1 in a wavelength range of 200 nm to 400 nm is the spectrum illustrated in FIG. 5.

As illustrated in FIG. 7, in a range where the sealing pressure ratio (P_{Cl}/P_{Kr}) is 2% or more, the light intensity integral in a wavelength range of 280 nm or more and less than 320 nm falls below 0.01 and hardly changes with respect to the change in the sealing pressure ratio (P_{Cl}/P_{Kr}). That is, according to FIG. 7, the relative intensity falls below 0.01 around the sealing pressure ratio (P_{Cl}/P_{Kr}) of 1.5%, but there is a risk that the relative intensity exceeds 0.01 due to manufacturing variations and the like. In contrast, in a case where the sealing pressure ratio (P_{Cl}/P_{Kr}) is 2.0% or more, a risk that the relative intensity exceeds 0.01 is extremely small even in consideration of manufacturing variations and the like.

As shown in FIGS. 8A and 8B, in the intensity spectrum of the excimer lamp 30 of Example 1, as compared with the intensity spectrum of the excimer lamp 30 of Comparative Example 1, the relative intensity is high in a wavelength range of 240 nm or more and less than 280 nm, and the relative intensity is low in a wavelength range of 280 nm or more and less than 400 nm.

The above results indicate that, as the sealing pressure ratio (P_{Cl}/P_{Kr}) of the luminous gas G1 sealed in the luminous tube 30a increases, the relative intensity of the ultraviolet light Lx generated in the luminous tube 30a within a wavelength range of 240 nm or more and less than 280 nm increases, and the relative intensity within a wavelength range of 280 nm or more and less than 400 nm decreases.

That is, it is confirmed that the relative intensity of the ultraviolet light Lx generated in the luminous tube 30a within a wavelength range of 240 nm or more and less than 400 nm can be controlled by adjusting the sealing pressure ratio (P_{Cl}/P_{Kr}) of the luminous gas G1 sealed in the luminous tube 30a.

Here, as described above, in many excimer lamps, in order to prevent a person from being irradiated with ultraviolet light in a wavelength band harmful to the human body, an optical filter that does not substantially transmit light in the wavelength band is combined.

In addition, in the dielectric multilayer film filter exhibiting the transmittance characteristics as illustrated in FIG. 6A, an increase in transmittance from around a wavelength of 280 nm toward the long wavelength side is confirmed. In a case where such a dielectric multilayer film filter is used, there is a high possibility that the ultraviolet light on the longer wavelength side than a wavelength of 300 nm becomes a problem more than the relative intensity at a wavelength of 240 nm or more and less than 280 nm.

Therefore, it is considered that the sealing pressure ratio (P_{Cl}/P_{Kr}) between krypton gas and chlorine gas contained in the luminous gas G1 is preferably adjusted to cause the relative intensity of ultraviolet light having a wavelength longer than 300 nm to be reduced.

In addition, during the execution of the present verification experiment, there was confirmed a phenomenon in which, as the sealing pressure ratio of the luminous gas G1 to be sealed in the luminous tube 30a of the excimer lamp 30 was increased, the excimer lamp became difficult to be turned on at the point where the sealing pressure ratio exceeded 5%. Halogen gas such as chlorine has high electronegativity and high electron adhesion. Therefore, when the pressure ratio of the chlorine gas in the luminous tube 30a increases, electrons in the luminous tube 30a easily adhere to chlorine, and the number of electrons required for discharge decreases. It is presumed that the phenomenon described above is caused by the decrease of electrons in the luminous tube 30a as described above. From the above finding, the sealing pressure ratio (P_{Cl}/P_{Kr}) between krypton gas and chlorine gas contained in the luminous gas G1 is desirably less than 5%.

Note that, because the electron adhesion of chlorine gas also contributes to the stability of the discharge column in the luminous tube 30a and the electrical load given to the lamp, if the amount of chlorine gas is too small with respect to krypton gas, discharge generated in the luminous tube 30a is destabilized and the life of the lamp is shortened. In comprehensively judging from the above viewpoints and the result in FIG. 7, it is found that the sealing pressure ratio (P_{Cl}/P_{Kr}) between krypton gas and chlorine gas contained in the luminous gas G1 is preferably 2% or more and less than 5%.

According to the above configuration, regarding the relative intensity with respect to the peak intensity of ultraviolet light emitted through the optical filter 20b, the intensity of ultraviolet light on the longer wavelength side than the main emission wavelength band can be reduced without greatly increasing the intensity of ultraviolet light in the wavelength range including the wavelength of 240 nm or more and less than 280 nm, which a wavelength band harmful to the human body. That is, the intensity of ultraviolet light in the wavelength band harmful to the human body can be maintained or reduced while the light intensity of ultraviolet light in the main emission wavelength band belonging to the wavelength band of 190 nm or more and less than 240 nm is increased.

Note that the characteristics of the ultraviolet light Lx emitted from the excimer lamp 30 shown in the above verification is considered to theoretically appear in an excimer lamp in which a luminous gas containing the noble gas and the halogen gas is sealed in a luminous tube. In particular, a similar feature is confirmed in an excimer lamp in which the luminous gas G1 containing krypton gas and bromine (Br) gas is sealed in the luminous tube 30a and which emits ultraviolet light having a main emission wavelength of around 207 nm.

That is, the inactivation apparatus 1 of the present invention may be equipped with an excimer lamp in which the luminous gas G1 containing the noble gas and the halogen gas is sealed in the luminous tube 30a, and as a specific example, the excimer lamp 30 can be employed in which the luminous gas G1 containing krypton gas and bromine gas is sealed in the luminous tube 30a.

In addition, in a case where the intensity is sufficiently reduced to such an extent that there is no problem even if the ultraviolet light having a wavelength of 280 nm or more and less than 400 nm is emitted as it is by adjusting the luminous gas G1 sealed in the luminous tube 30a of the excimer lamp 30, an optical filter having a band in which the ultraviolet light is transmitted within a wavelength range of 280 nm or more and less than 320 nm may be adopted.

### Second Embodiment

A configuration of a second embodiment of an inactivation apparatus 1 according to the present invention is described, centering on features different from the first embodiment.

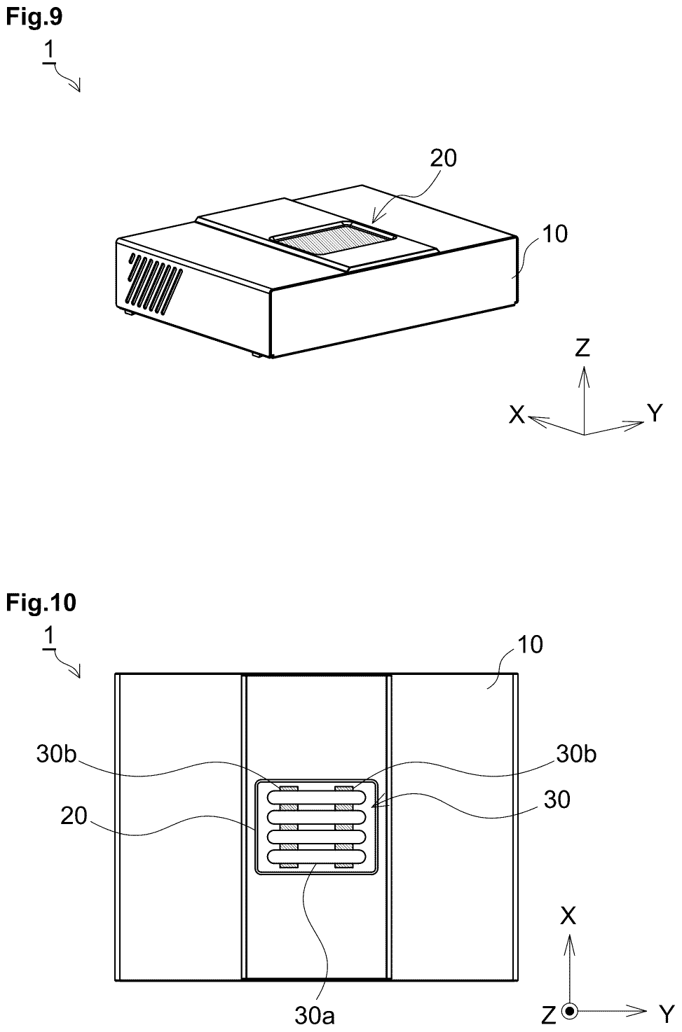

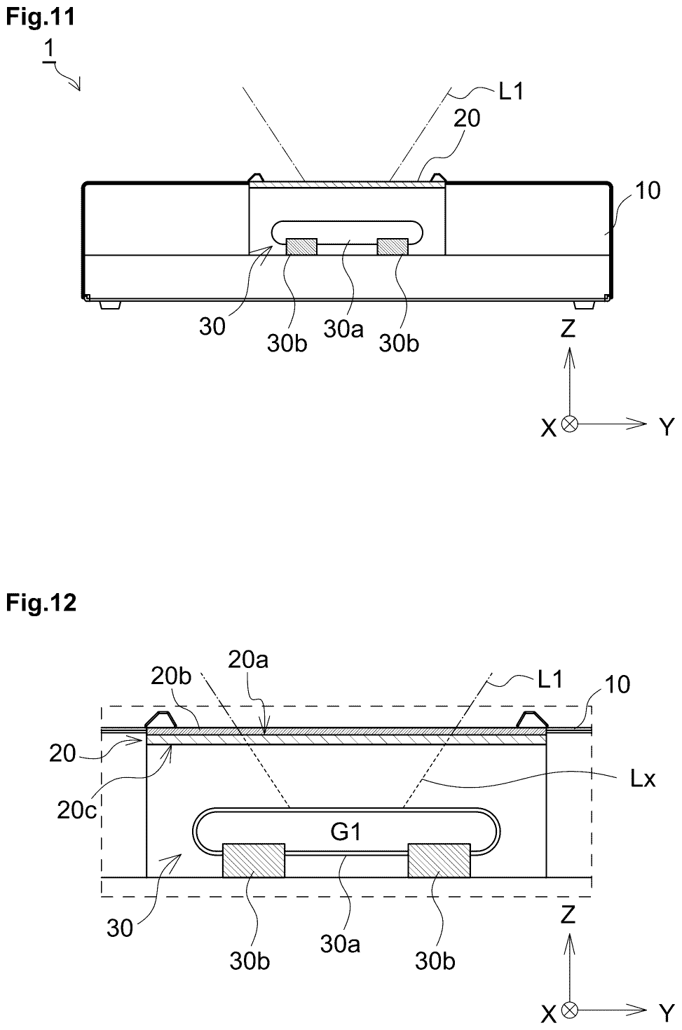

FIG. 9 is a schematic view illustrating an external appearance of the inactivation apparatus 1 according to the second embodiment, and FIG. 10 is a drawing of the inactivation apparatus 1 in FIG. 9 as viewed from +Z side. FIG. 11 is a cross-sectional view of the inactivation apparatus 1 in FIG. 9 as viewed in the X direction, and FIG. 12 is an enlarged view of the periphery of an excimer lamp 30 in FIG. 11.

In the second embodiment of the inactivation apparatus 1, a use mode is assumed in which the inactivation apparatus 1 is placed on a table or the like and the ultraviolet light is emitted toward the inside of a predetermined partitioned space.

As illustrated in FIG. 9, the excimer lamp 30 of the second embodiment is an excimer lamp including a plurality of luminous tubes 30a and a pair of electrodes 30b. As illustrated in FIG. 10, the plurality of luminous tubes 30a are placed on the pair of electrodes 30b.

As illustrated in FIG. 11, an optical filter 20b of the second embodiment is formed on a principal surface 20a of a light transmission window 20. The configuration is the same as that of the first embodiment, but in the second embodiment, a length of the luminous tube 30a of the excimer lamp 30 in the tube axis direction (Y direction) is 70 mm, a distance between the excimer lamp 30 and the optical filter 20b is 8 mm, and a size of the optical filter 20b is (X, Y)=(60 mm, 45 mm).

The configurations included in the inactivation apparatus 1 described above are merely examples, and the present invention is not limited to the illustrated configurations.

### DESCRIPTION OF REFERENCE SIGNS

1: Inactivation apparatus
10: Housing
20: Light transmission window
20a: Principal surface
20b: Optical filter
20c: Principal surface
30: Excimer lamp
30a: Luminous tube
30b: Electrode
G1: Luminous gas
L1, Lx: Ultraviolet light

1. An inactivation apparatus comprising:
an excimer lamp that includes a luminous tube in which a luminous gas containing a noble gas and a halogen gas is sealed, and a pair of electrodes, and generates ultraviolet light having a main emission wavelength band within a range of 190 nm or more and less than 240 nm in the luminous tube when a voltage is applied between the pair of electrodes; and
an optical filter into which ultraviolet light generated by the excimer lamp is incident, the optical filter transmitting ultraviolet light having a wavelength within a range of 190 nm or more and less than 240 nm and substantially not transmitting ultraviolet light having a wavelength within a range of 240 nm or more and less than 280 nm, wherein
the luminous gas sealed in the luminous tube has a ratio of a sealing pressure of the halogen gas to a sealing pressure of the noble gas of 2% or more and less than 5%.

2. The inactivation apparatus according to claim 1, wherein the optical filter has a band that transmits ultraviolet light in at least a part of a range of a wavelength of 280 nm or more and less than 320 nm.

3. The inactivation apparatus according to claim 1, wherein the luminous gas is a mixed gas containing krypton (Kr) and chlorine (Cl).

4. The inactivation apparatus according to claim 1, wherein the luminous gas is a mixed gas containing krypton (Kr) and bromine (Br).

5. The inactivation apparatus according to claim 2, wherein the luminous gas is a mixed gas containing krypton (Kr) and chlorine (Cl).

6. The inactivation apparatus according to claim 2, wherein the luminous gas is a mixed gas containing krypton (Kr) and bromine (Br).

7. The inactivation apparatus according to claim 1, wherein the luminous gas contains argon (Ar) gas as a buffer gas.

8. The inactivation apparatus according to claim 1, further comprising:
a housing that the excimer lamp is accommodated; and
a light transmission window that the ultraviolet light emitted from the excimer lamp is extracted to the outside of the housing, wherein
the optical filter is formed on the principal surface located inside the housing of the light transmission window.

9. The inactivation apparatus according to claim 1, further comprising:
a housing that the excimer lamp is accommodated; and
a light transmission window that the ultraviolet light emitted from the excimer lamp is extracted to the outside of the housing, wherein
the optical filter is formed on the principal surface located outside the housing of the light transmission window.
